SEQUENCE LISTING (4 sequences)

SEQ ID NO: 1 - DNA, 36 bp, Homo Sapiens
  modified_base (18)..(18): 5hmC
tcgatgtagtgcgtcaccggatgatagctgtactca

SEQ ID NO: 2 - DNA, 36 bp, homo sapiens
  modified_base (18)..(18): 5-methylcytosine
tgagtacagctatcatccggtgacgcactacatcga

SEQ ID NO: 3 - DNA, 36 bp, Artificial Sequence; identical to SEQ ID NO: 2
  single-stranded DNA probe

SEQ ID NO: 4 - DNA, 36 bp, Artificial Sequence; identical to SEQ ID NO: 1
  single-stranded DNA probe

(19) United States
(12) Patent Application Publication
(10) Pub. No.: US 2024/0159742 A1
(43) Pub. Date: May 16, 2024

(54) EPIGENETIC QUANTIFICATION USING DNA HYBRIDIZATION-BASED SINGLE-MOLECULE IMMUNOFLUORESCENT IMAGING

(71) Applicant: University of Cincinnati, Cincinnati, OH (US)

(72) Inventor: Jiajie Diao, Lebanon, OH (US)

(73) Assignee: University of Cincinnati, Cincinnati, OH (US)

(21) Appl. No.: 18/280,513

(22) PCT Filed: Mar. 10, 2022
(86) PCT No.: PCT/US2022/019704

§ 371 Date: Sep. 6, 2023

Related U.S. Application Data
(60) Provisional application No. 63/159,064, filed on Mar. 10, 2021.

(51) Int. Cl. G01N 33/53; G01N 33/574; G01N 33/58; G06T 7/00

(52) U.S. Cl. CPC G01N 33/5308; G01N 33/57484; G01N 33/582; G06T 7/0012; G06T 2207/10056; G06T 2207/10064; G06T 2207/30024; G06T 2207/30096

(57) ABSTRACT

Disclosed herein is a method of epigenetic quantification using DNA hybridization-based single-molecule immunofluorescent imaging, an ultra-sensitive method of detecting epigenetic modifications in DNA. Via use of probe DNA to capture the DNA fragment of interest and the immunofluorescent imaging to detect modifications, the fluorescent response signal can be detected and quantified at the single-molecule level.

### CROSS REFERENCE TO RELATED APPLICATION

This application claims priority to U.S. Provisional Application Ser. No. 63/159,064, filed Mar. 10, 2021, the entire disclosure of which is hereby incorporated by reference.

### TECHNICAL FIELD

The present invention relates to quantification of epigenetic modifications using DNA hybridization-based single-molecule immunofluorescent imaging.

### SEQUENCE LISTING

Applicant incorporates by reference a CRF sequence listing submitted herewith having file name Sequence_Listing_10738_931.txt, created on Mar. 3, 2022.

The nucleic acid sequences listed in the accompanying sequence listing are shown using standard abbreviations as defined in 37 C.F.R. § 1.822. In the accompanying sequence listing:
SEQ ID NO: 1 represents a target DNA strand having one 5hmC modification;
SEQ ID NO: 2 represents a non-target DNA strand having one 5mC modification;
SEQ ID NO: 3 represents a single-stranded DNA probe complementary to a target DNA strand;
SEQ ID NO: 4 represents a single-stranded DNA probe complementary to a non-target DNA strand.

### BACKGROUND

DNA epigenetic modifications play important functions in a broad range of physiological and pathological processes and their dysregulation can lead to various human diseases. 5-hydroxymethylcytosine (5hmC), one of the major mammalian DNA epigenetic modifications, is generated by ten-eleven translocation (TET) family proteins from 5-methylcytosine (5mC) and is often referred to as the sixth base of DNA, due to its involvement in epigenetic reprogramming and regulation of gene expression. 5hmC is tissue-specific and is believed to be a gene activation marker in development and disease. Since its discovery in neurons in 2009, 5hmC has been shown to play critical roles in embryonic development and human diseases. Recently, 5hmC has been reported as an epigenetic biomarker for several types of cancer.

To detect and quantify 5hmC in DNA, several methods have been developed using 5hmC antibodies, but these methods are limited because they lack information about which gene contains the epigenetic modification. Meanwhile, by converting unmethylated cytosine to uracil, other researchers have shown that bisulfite sequencing and its derived methods can fully profile 5mC and 5hmC in DNA. These methods are limited, as bisulfite treatments degrade DNA. This degradation means that such a method requires large quantities of DNA, however, in humans, 5hmC occurs at a relatively low frequency compared to other epigenetic modifications. Many methods for quantifying 5hmC, including TAB-seq, oxBS-seq, hMeSeal-seq, and hme-DIP require at least 5 ng of DNA.

Circulating cell-free DNA (cfDNA) are short, degraded nucleic acid fragments in circulation in the bloodstream. The non-invasive availability of cfDNA makes it a promising biomarker for diagnosing, prognosing, and monitoring tumor evolution and response to therapy. Using a sensitive chemical labeling-based low-input sequencing method, the present investigators previously conducted rapid and reliable sequencing of 5hmC in cfDNA and showed that cell-free 5hmC displays distinct features in several types of cancer. Song, et al., 5-Hydroxymethylcytosine signatures in cell-free DNA provide information about tumor types and stages, Cell Res. 27(10): 1231-42 (2017). These findings have potential application not only in identifying cancer types, but also in diagnosis of cancer and tracking tumor stage in some cancers. In order to work with the minute quantities of cfDNA available (typically only a few nanograms per ml of plasma), ultra-sensitive detection methods are required for diagnosing early stage cancers.

Single-molecule optical detection has increasingly become an attractive and competitive tool for analytical epigenetics in view of its extreme sensitivity and inherent multiplexing, as well as its potential utility for cost-effective diagnostic applications. Ultra-sensitive single-molecule epigenetic imaging for quantifying and identifying interactions between 5hmC and 5mC have been previously described. See Song, et al., Simultaneous single-molecule epigenetic imaging of DNA methylation and hydroxymethylation, PNAS 113(16): 4338-43 (2016); US 20170298422. However, current methods of single-molecule epigenetic imaging are still blind to the specific genomic location of epigenetic modifications, which information provides additional insight to the diagnosing practitioner.

The numerous methods that have been developed to detect and quantify 5hmC require large amounts of DNA sample to be modified via chemical reactions, which considerably limits their application with cell-free DNA (cfDNA).

Accordingly a need exists for a sensitive, low-input assay of quantifying 5hmC in DNA fragments at the single-molecule level.

### SUMMARY

Accordingly, described herein is a method for epigenetic quantification using DNA hybridization based single-molecule immunofluorescent imaging for detection and quantification of DNA epigenetic modifications.

In one embodiment, a method for quantifying epigenetic modifications in DNA is provided, the method comprising: providing a target DNA strand comprising a least one epigenetic modification, annealing a single-stranded DNA probe to the target DNA strand, wherein the probe is conjugated to a biotin moiety; immobilizing the annealed DNA on a support; contacting the immobilized DNA with a primary antibody that binds to the at least one epigenetic modification; contacting the immobilized DNA with a secondary antibody, wherein the second antibody is labeled with a fluorophore and wherein the secondary antibody binds to the primary antibody; and detecting the fluorophore using prism-based single molecule total internal reflection fluorescence (TIRF) microscopy.

In another embodiment, a method of diagnosing cancer in a subject suspected of having cancer is provided, the method comprising: providing a biological sample from the subject, the sample comprising a target DNA strand comprising a least one epigenetic modification, wherein the target DNA strand is annealed to a non-target DNA strand; annealing a single-stranded DNA probe to the target DNA strand, wherein the probe is conjugated to a biotin moiety; immobilizing the annealed DNA on a support; contacting the immobilized DNA with a primary antibody that binds to the epigenetic modification; contacting the immobilized DNA with a secondary antibody, wherein the second antibody is labeled with a fluorophore and wherein the secondary antibody binds to the primary antibody; detecting the fluorophore using prism-based single molecule total internal reflection fluorescence (TIRF) microscopy based on the fluorophore detection; quantifying a number of epigenetic modifications in the target DNA strand; comparing the number of epigenetic modifications to a reference epigenetic profile for cancer; and diagnosing the subject as having cancer when the quantifying correlates with the reference epigenetic profile for cancer.

These and other features and benefits of the various embodiments of the present invention will become apparent from the following description, which includes figures and examples of specific embodiments intended to give a broad representation of the invention. Various modifications will be apparent to those skilled in the art from this description and from practice of the invention. The scope is not intended to be limited to the particular forms disclosed and the invention covers all modifications, equivalents and alternatives falling within the spirit and scope of the invention as defined by the claims.

### BRIEF DESCRIPTION OF THE DRAWINGS

The following detailed description of specific embodiments of the present disclosure can be best understood when read in conjunction with the following drawings:

FIG. 1 depicts an embodiment of a DNA hybridization step of a method of DNA hybridization based single-molecule epigenetic quantitation of 5hmC. A target DNA strand (TS), containing 5hmC, is annealed with a non-target DNA strand (NTS), containing 5mC, both of which are 3′ end labeled with Cy3. A single strand DNA probe (SP) is 3′ end labeled with biotin and anneals to the TS.

FIG. 2 schematically depicts an embodiment of a method of DNA hybridization based single-molecule immunofluorescent imaging of 5hmC wherein the annealed DNA from FIG. 1 is immobilized on a support, treated with primary and secondary antibodies and imaged with single-molecule total internal reflection fluorescence (TIRF) microscopy.

FIG. 3A shows that, before annealing, Cy3 and Alexa 647 cannot be observed, because the DNA is not immobilized. After annealing Cy3 can be detected for both TS/SP and NTS/CSP annealed samples, indicating that annealed DNA is immobilized on the support. Alexa 647 is observed in the sample annealing TS and SP, indicating that the method successfully detects 5hmC.

FIG. 3B depicts the quantitative representation of the fluorophore counts depicted in FIG. 3A.

FIGS. 4A-4B depict FRET histograms showing high FRET for the TPA group (FIG. 4B) compared to the TnPA (FIG. 4A).

FIG. 5A depicts representative images of 5hmC signals (Alexa 647 fluorophore counts) in different concentrations of freshly annealed dsDNA.

FIG. 5B depicts the quantitative representation of the fluorophore counts depicted in FIG. 5A.

FIG. 5C depicts representative images of 5hmC signals (Alexa 647 fluorophore counts) when incubating with different concentrations of 5hmC primary antibody.

FIG. 5D depicts the quantitative representation of the fluorophore counts depicted in FIG. 5C.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided herein. The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

While the following terms are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, a “subject” refers to a mammalian subject. Optionally, a subject is a human or non-human primate. Optionally, the subject is selected from the group consisting of mouse, rat, rabbit, monkey, pig, and human. In a specific embodiment, the subject is a human.

The terms “treat,” “treatment,” and “treating,” as used herein, refer to a method of alleviating or abrogating a disease, disorder, and/or symptoms thereof in a subject.

An “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

“Epigenetic modification,” as used herein, refers to modifications of the genome that are heritable, but that do not involve alterations of nucleotide sequence. Epigenetic modifications may be associated with gene activity and expression, or may contribute to other phenotypic traits. Various epigenetic modifications are known, including DNA methylation, RNA modification, and histone modification, which alter how a gene is expressed without modifying the underlying nucleotide sequence. The presently disclosed methods are suitable for detection of epigenetic modifications comprising, for example, methylation of nucleic acids. Epigenetic modifications of DNA detectable by the present methods include, for example, 5-hydroxymethylcytosine (5hmC), 5-methylcytosine (5mC), 5-formylcytosine (5fC), 5-carboxylcytosine (5caC), and the like. Epigenetic modifications of RNA detectable by the present methods include, for example, as N6-methyladenosine (m6A).

“Genomic DNA (gDNA),” as used herein, refers to chromosomal DNA that carries biological information of heredity passed from one generation to the next.

“Target DNA strand (TS),” as used herein, refers to a coding DNA strand of interest that comprises at least one epigenetic modification. “Non-target DNA strand (NTS),” as used herein, refers to a DNA strand that may be annealed to the target DNA strand.

The terms “anneal” and “hybridize” are used interchangeably herein and refer to the phenomena by which complementary nucleic acid strands pair via hydrogen bonding to form a double-stranded polynucleotide. If two nucleic acids are “complementary,” each base of one of the nucleic acids base pairs with corresponding nucleotides in the other nucleic acid. Two nucleic acids need not be perfectly complementary in order to hybridize to one another.

“Biological sample,” as used herein, refers to a clinical sample obtained from a subject for use in the present methods. In embodiments, the biological sample comprises nucleic acids, such as target DNA and/or non-target DNA. In particular embodiments, the biological sample is selected from cells, tissues, bodily fluids, and stool. Bodily fluids of interest include, but are not limited to, blood, serum, plasma, saliva, mucous, phlegm, cerebral spinal fluid, pleural fluid, tears, lactal duct fluid, lymph, sputum, synovial fluid, urine, amniotic fluid, and semen. In a specific embodiment, the biological sample is selected from the group consisting of blood, serum, plasma, urine, tissue, and cultured cells.

“Total internal reflection fluorescence (TIRF) microscopy,” as used herein, refers to a method of microscopy that permits imaging of a thin region of a specimen by exploiting unique properties of an induced evanescent wave or field in a limited specimen region immediately adjacent to the interface between two media having different refractive indices (for example, the contact area between a specimen and a glass coverslip or tissue culture container). Visualization of single-molecule fluorescence with sufficient temporal resolution for dynamic studies is possible with TIRF because of the high signal-to-noise ratio afforded by the evanescent wave excitation.

“Avidin-biotin pairing,” as used herein, refers to an affinity tag pair wherein a first member of the pair is a biotin moiety, and a second member of the pair is selected from the group consisting of avidin, streptavidin, and neutravidin or other modified form of avidin.

As used herein, the term “biotin moiety” refers to an affinity tag that includes biotin or a biotin analogue such as desthiobiotin, oxybiotin, 2-iminobiotin, diaminobiotin, biotin sulfoxide, biocytin, etc.

As used herein, the term “support” refers to a support (e.g., a planar support such as a microscope slide) that binds biotin or a biotin moiety. In embodiments, the support is linked to avidin, streptavidin, or neutravidin or other modified form of avidin. In a specific embodiment, the support is a polymer-coated quartz surface.

“Localizing” and “localization,” as used herein, refer to determining the location of an epigenetic modification on a target DNA strand. In embodiments, the disclosed methods permit strand-specific and/or loci-specific localization of discrete epigenetic modifications of genomic and cf DNA, such as 5hmC, 5mC, and the like.

Disclosed herein is a method for quantifying epigenetic modifications using DNA hybridization based single-molecule immunofluorescent imaging (SMII), a single-molecule optical detection-based method for loci-specific and strand-specific epigenetic modification imaging as well as quantification. SMII achieves ultrasensitivity and is applied herein to image genomic DNA and cfDNA to demonstrate its utility and clinical application.

In one embodiment, a method for quantifying epigenetic modifications of DNA is provided, the method comprising: (a) providing a target DNA strand comprising at least one epigenetic modification; (b) annealing a single-stranded DNA probe to the target DNA strand, wherein the probe is conjugated to a biotin moiety; (c) immobilizing the annealed DNA on a support; (d) contacting the immobilized DNA with a primary antibody that binds to the at least one epigenetic modification; (e) contacting the immobilized DNA with a secondary antibody, wherein the second antibody is labeled with a fluorophore and wherein the secondary antibody binds to the primary antibody; and (f detecting the fluorophore using prism-based single molecule total internal reflection fluorescence (TIRF) microscopy.

DNA strands may include genomic DNA and/or cfDNA from a eukaryotic source, including, but not limited to, plants, animals (e.g., reptiles, mammals, insects, worms, fish, etc.), fungi (e.g., yeast), and the like, as well as genomic DNA isolated from tissue samples. In certain embodiments, the DNA used in the disclosed method is derived from a biological sample obtained from mammal, such as a human.

In some embodiments, the biological sample is obtained from a subject that has or is suspected of having a disease or condition associated with epigenetic modifications, such as a cancer, inflammatory disease, or pregnancy. In some embodiments, the biological sample may be made by extracting fragmented DNA from a fresh or archived patient sample, e.g., a formalin-fixed paraffin embedded tissue sample. In other embodiments, the biological sample may be a sample of cfDNA from a bodily fluid, e.g., peripheral blood.

The DNA used in the initial steps of the method comprises non-amplified DNA and, in certain embodiments, has not been denatured beforehand. In embodiments, the DNA is fragmented for use in the instant methods. DNA may be fragmented mechanically (e.g., by sonication, nebulization, or shearing) or enzymatically, using a double-stranded DNA fragmentase enzyme (New England Biolabs, Ipswich MA). In other embodiments, the DNA in the initial sample may already be fragmented (e.g., as is the case for FFPE samples and cfDNA, e.g., ctDNA (circulating tumor DNA)).

In some embodiments, the fragments in the initial sample may have a median size that is below 1 kb (e.g., in the range of 50 bp to 500 bp, 80 bp to 400 bp, or 100-1,000 bp), although fragments having a median size outside of this range may be used. Cell-free or circulating tumor DNA (ctDNA), i.e., tumor DNA circulating freely in the blood of a cancer patient, is highly fragmented, with a mean fragment size about 165-250 bp. cfDNA can be obtained by centrifuging whole blood to remove all cells, and then analyzing the remaining plasma.

Various fluorophores are known in the art and suitable for use in the present methods. Suitable distinguishable fluorescent label pairs for use in the disclosed methods include, but are not limited to, Cy-3 and Cy-5 (Amersham Inc., Piscataway, N.J.), Quasar 570 and Quasar 670 (Biosearch Technology, Novato, CA), Alexa Fluor 555 and Alexa Fluor 647 (Molecular Probes, Eugene, OR), BODIPY V-1002 and BODIPY V-1005 (Molecular Probes, Eugene, OR), POPO-3 and TOTO-3 (Molecular Probes, Eugene, OR), PO-PRO3 TO-PRO3 (Molecular Probes, Eugene, OR), and the like. In embodiments, first and second fluorophores may be used to differentiate between different epigenetic modifications. In such embodiment, first and second fluorophores are optically-distinguishable, such that moieties labeled with first and second fluorophores can be independently detected. Further suitable distinguishable detectable labels may be found in Kricka, Stains, labels and detection strategies for nucleic acid assays, Ann. Clin. Biochem. 39(2): 114-29, (2002).

In embodiments, each of the target DNA strand and the non-target DNA strand are end-labeled at a 3′ end with a fluorophore. Methods of end-labeling DNA are known in the art, and include, for example, terminal transferase reactions. In embodiments, such as when dsDNA is collected from a subject, the DNA may not be end-labeled. In embodiments, the dsDNA may be labeled prior to hybridization.

In embodiments, the at least one epigenetic modification is selected from the group consisting of 5-hydroxymethylcytosine (5hmC), 5-methylcytosine (5mC), 5-formylcytosine (5fC), and 5-carboxylcytosine (5caC). In a specific embodiment, the at least one epigenetic modification comprises 5hmC.

The present methods utilize a probe, wherein a single-stranded DNA probe is designed to be complementary to the target DNA strand. In embodiments, a second single-stranded DNA probe is designed to be complementary to the non-target DNA strand. In embodiments, the first and second probes are complementary to each other.

The ratio of first probe to target DNA strand is selected to provide an excess of probe, in order to facilitate capture of as much target DNA as possible. In embodiments, the ratio of first probe to target DNA strand is about 10:1, about 100:1, about 1000:1, or about 10,000:1. In a specific embodiment, the ratio of first probe to target DNA is about 100:1.

The probes are labeled with a biotin moiety to enable capture on a suitable support, which is correspondingly labeled with a surface-tethered moiety that binds a biotin moiety. In embodiments, the probes are labeled with biotin and the support comprises a surface-tethered moiety selected from the group consisting of avidin, streptavidin, and neutravidin. In a specific embodiment, the surface-tethered moiety is neutravidin. In this way, target DNA strands may be captured and immobilized on a support via avidin-biotin pairing.

To prepare DNA, dsDNA fragments containing the target DNA strand are mixed with a corresponding single-stranded DNA probe in different molar ratios under annealing conditions. For example, dsDNA fragments are mixed with corresponding single-stranded DNA probes in annealing buffer, heated to denature the dsDNA fragments, and then cooled to facilitate annealing of the target DNA strand with the probe. The concentration of annealed DNA can be varied by adjusting the concentrations of the dsDNA containing the targeted strand and corresponding probe ssDNA with a constant 100:1 ratio of dsDNA to probe ssDNA. The newly annealed DNA (fluorophore-labeled and conjugated with a biotin moiety) is then ready for immobilization and imaging.

Immobilizing labeled DNA molecules on a support, such as a microscope slide, is accomplished using a slide coated in a binding partner for the capture tag added to the DNA molecules. For example, in some embodiments, DNA molecules labeled with a biotin moiety may be captured on a slide coated in avidin, streptavidin, or neutravidin. These slides may be made by first passivating the slides in a mixture of polyethylene glycol (PEG) mPEG-SVA and biotin-PEG-SVA (at a ratio of, e.g., 99:1 (mol/mol)) to reduce non-specific binding of the DNA, and then coating the slide in avidin, streptavidin, or neutravidin. In embodiments, additional blocking steps may be performed prior to coating the slide with avidin. The blocking buffer may be any acceptable blocking buffer known in the art. In embodiments, the blocking buffer may contain polysorbate 20 and/or serum. The labeled DNA molecules can be immobilized on the surface of the slide, e.g., at a concentration of 1-500 pM (e.g., 30-100 pM) for a period of time, e.g., 5 minutes to 1 hour, e.g., 30 minutes. The support is washed to remove unbound DNA.

After the newly annealed DNA molecules are immobilized, the slides can be blocked, using any acceptable blocking buffer. Primary antibodies corresponding to the epigenetic modification in the target DNA strand are applied to the slides and incubated for an appropriate amount of time, e.g. 5 min to 1 hour, e.g. 30 minutes. In embodiments, the primary antibodies are selected from anti-5hmC, anti-5mC, anti-5fC, anti-5caC, and the like. Fluorophore-labeled secondary antibodies, capable of binding to the primary antibodies, are applied to the slides and incubated for an appropriate amount of time, e.g. 5 min to 1 hour, e.g. 15 minutes.

Individual molecules of epigenetically modified DNA are imaged on the support at a single-molecule resolution. Imaging may employ any sensitive, high resolution, fluorescence detector equipped to excite the fluorophores. Appropriate filters should be used so that the signals from the first and second fluorophores can be separately detected and imaged. In one embodiment, the imaging employs total internal reflection fluorescence (TIRF) microscopy. For TIRF microscopy, a dual-laser excitation system is used to excite each of the first and second fluorophores. Total fluorescence signals from first and second fluorophores are collected by a water immersion objective lens and passed through a notch filter to block excitation beams. Emission signals from the second fluorophore (i.e., the epigenetic modification(s)) are separated by a dichroic mirror and detected by an electron-multiplying charge-coupled device camera. Data are recorded to provide fluorescence intensity signal and/or time trajectories of individual molecules.

After the labeled DNA molecules have been imaged, the method may further comprise counting the number of individual labeled DNA molecules, thereby determining the number of epigenetically modified DNA molecules in the sample.

Imaging provides loci-specific and/or strand-specific localization of at least one epigenetic modification of the DNA.

“Epigenetic profile,” as used herein, refers to a loci-specific and strand-specific epigenetic modification signature determined by the instant methods for a given DNA sample. In embodiments, the “reference epigenetic profile” for cancer or for a particular type of cancer is determined by carrying out the disclosed methods on one or more control samples. Loci- and strand-specific epigenetic modification data is collected from the reference population to provide a reference epigenetic profile. In embodiments, the control is an external control, such that imaging data obtained from the subject to be diagnosed is compared to imaging data from individuals known to suffer from, or known to be at risk of, a given condition (i.e., the reference population). In other embodiments, the imaging data obtained from the subject to be diagnosed is compared to imaging data from normal, healthy individuals. It should be understood that the reference population may consist of approximately 20, 30, 50, 200, 500 or 1000 individuals, or any value therebetween.

In some embodiment, the different samples may consist of an “experimental” sample, i.e., a sample of interest, and a “control” sample to which the experimental sample may be compared. In embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

The methods described above may be used to identify an epigenetic modification signature, or profile, that correlates with phenotype, e.g., a disease, condition or clinical outcome, etc. In some embodiments, this method may comprise (a) performing the above-described method on a plurality of DNA samples, wherein the DNA samples are isolated from patients having a known phenotype, e.g., disease, condition or clinical outcome, thereby determining a signature of epigenetic modification in DNA from each of the patients; and (b) identifying an epigenetic profile that is correlated with the phenotype.

In some embodiments, the epigenetic profile may be diagnostic (e.g., may provide a diagnosis of a disease or condition or the type or stage of a disease or condition, etc.), prognostic (e.g., indicating a clinical outcome, e.g., survival or death within a time frame), or theranostic (e.g., indicating which treatment would be the most effective).

Also provided is a method for analyzing a patient sample. In this embodiment, the method may comprise: (a) identifying, using the above-described method, an epigenetic profile in the DNA of a patient; (b) comparing the identified sequences to a reference epigenetic profile that correlates with a phenotype, e.g., a disease, condition, or clinical outcome etc.; and (c) providing a report indicating a correlation with phenotype. This embodiment may further comprise making a diagnosis, prognosis or theranosis based on the results of the comparison. It should be understood that the present methods are applicable to a wide range of diseases, conditions, or clinical outcomes characterized by epigenetic modifications to nucleic acids.

In a specific embodiment, the method comprises (a) providing a biological sample from the subject, the sample comprising a target DNA strand comprising a least one epigenetic modification, wherein the target DNA strand is annealed to a non-target DNA strand; (b) annealing a single-stranded DNA probe to the target DNA strand, wherein the probe is conjugated to a biotin moiety; (c) immobilizing the freshly annealed DNA on a support; (d) contacting the immobilized DNA with a primary antibody that binds to the epigenetic modification; (e) contacting the immobilized DNA with a secondary antibody, wherein the second antibody is labeled with a fluorophore and wherein the secondary antibody binds to the primary antibody; (f detecting the first fluorophore using prism-based single molecule total internal reflection fluorescence (TIRF) microscopy; (g) quantifying a number of epigenetic modifications in the target DNA strand based on the fluorophore detection; (f) comparing the number of epigenetic modifications to a reference epigenetic profile for cancer; and (h) diagnosing the subject as having cancer when the quantifying of step (g) correlates with the reference epigenetic profile for cancer.

In embodiments, the subject is diagnosed with cancer when the subject's epigenetic profile is concordant with the reference epigenetic profile for cancer. In a specific embodiment, the subject is diagnosed with cancer when the subject's epigenetic profile is at least 80% concordant with the reference epigenetic profile.

“Concordant,” as used herein, refers to the degree of identity between compared datasets, including imaging, or epigenetic profile, datasets. In certain embodiments, concordant refers to at least 25%, at least 50%, at least 75%, at least 80%, at least 85%, at least 90%, at least 91%, at least 92%, at least 93%, at least 94%, at least 95%, at least 96%, at least 98%, at least 99%, or 100% identity.

In embodiments, the method further comprises treating the diagnosed patient with an effective amount of a therapeutic agent specific for the cancer diagnosed.

While cancer is an exemplary disease for application of the instant methods, it should be understood that the disclosed methods may be applied to any disease, condition, or clinical outcome characterized by epigenetic modifications to nucleic acids. Such diseases, conditions, or clinical outcomes may be assessed via SMEL using single-stranded probes designed to be complementary to known genomic regions having epigenetic modifications associated with said disease, condition, or clinical outcome.

In other embodiments, the presently disclosed methods are suitable for use in identifying epigenetic patterns or profiles of DNA from other species, including plant and animal species. For example, single-stranded probes designed to be complementary to known genomic regions having epigenetic modifications can be employed in the instant methods to rapidly determine a source of DNA.

### Examples

The following examples are given by way of illustration and are in no way intended to limit the scope of the present disclosure.

### Example 1. Materials and Methods

### Preparation of DNA Fragments for Single-Molecule Immunofluorescent Imaging

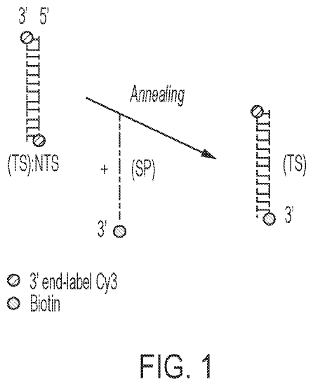

All of the single-stranded DNA probes with biotin at the 3′ end were obtained from Integrated DNA Technologies (IDT). To prepare DNA for immunofluorescent detection, dsDNA fragments, containing a target strand of DNA with a 5hmC epigenetic modification (SEQ ID NO: 1) (acquired from Prof. Chunxiao Song), were mixed with a biotin-labelled, single-stranded DNA probe (SEQ ID NO: 3) in annealing buffer (10 mM Tris, 1 mM EDTA, 50 mM NaCl, pH 8.0), heated for 5 min at 95° C. and cooled to room temperature (approximately 3-4 hours), as shown in FIG. 1. DNA sequence information can also be located in Table 1. Both the TS and the NTS were labeled on their 3′ end with Cy3.

TABLE 1
DNA sequence information.

| Name | Sequence (5′-3′) |
|---|---|
| TS (target DNA | TCGATGTAGTGCGTCACXGGAT |
| strand) | GATAGCTGTACTCA |
| (SEQ ID NO: 1) | |
| NTS (non-target | TGAGTACAGCTATCATCYGGTG |
| DNA strand) | ACGCACTACATCGA |
| (SEQ ID NO: 2) | |
| SP (ssDNA probe) | TGAGTACAGCTATCATCCGGTG |
| (SEQ ID NO: 3) | ACGCACTACATCGA-Biotin |
| CSP (Complementary | TCGATGTAGTGCGTCACCGGAT |
| ssDNA probe) | GATAGCTGTACTCA-Biotin |
| (SEQ ID NO: 4) | |

Underlined X represents 5hmC.
Underlined Y represents 5mC.

### Surface Passivation and Flow Chamber Construction

To prepare a support, quartz slides were sonicated in 1 M KOH, acetone and methanol for 30 minutes each. The slides were then burned for one minute. These slides, along with coverslips, were incubated in a mixture of methanol, acetic acid, and aminosilane for 20 minutes in the dark. During the incubation, slides and coverslips were sonicated once for 1 minute. The slides and coverslips were then coated with a mixture of 9700 mPEG (Laysan Bio) and 3% biotin PEG (Laysan Bio). The slides were immediately covered with coverslips and stored overnight in a humidified box. The next day, the slides and coverslips were washed with purified water (Milli-Q®) and dried using blown nitrogen. Flow chambers were assembled using the prepared slides and placing double sided tape between the slide and the coverslips to create channels. The edges were sealed using epoxy.

### Immobilization of DNA for Single-Molecule Imaging

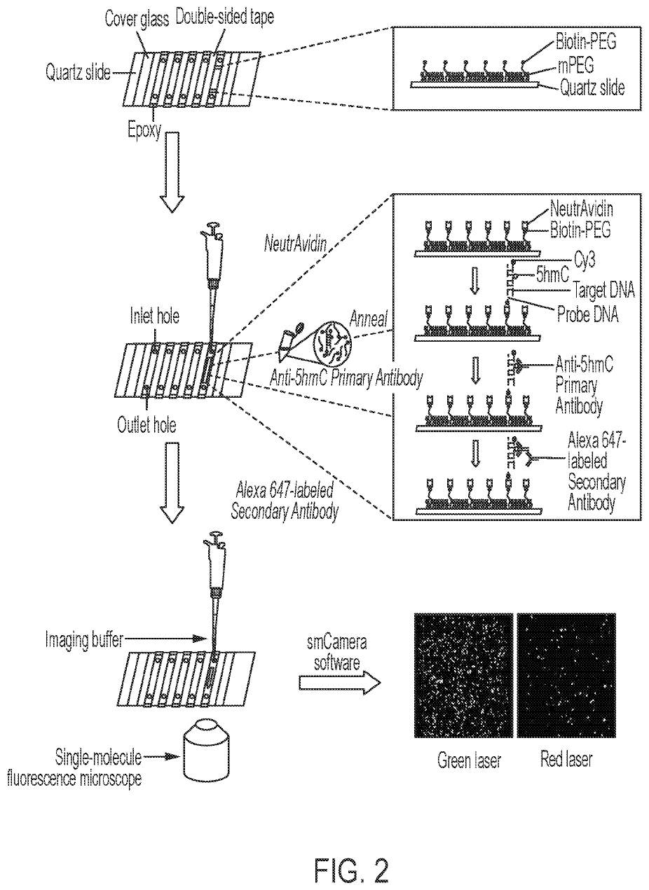

To immobilize DNA samples for immunofluorescent detection, the prepared slide was blocked with a solution containing 1% (v/v) polysorbate 20 (TWEEN® 20, ALKEST® TW 20, etc.) for 30 minutes. A 0.2 mg/ml solution of NeutrAvidin (Thermo Fisher Scientific) was prepared by diluting the neutravidin in T50 buffer (10 mM Tris Base and 50 mM NaCl, pH 8.0). 40 μl of the neutravidin solution was flowed into each flow chamber and incubated for 10 minutes. Excess neutravidin was washed away by flowing 200 μl T50 buffer through each flow chamber twice. 40 μl of the newly annealed DNA was injected into each flow chamber and incubated for 15 minutes. Unbound DNA was washed away by rinsing twice with 200 μl T50 buffer. The slides were then blocked with blocking buffer (10% v/v polysorbate 20 and 1% v/v serum) and incubated for 30 minutes. This is schematically shown in FIG. 2.

The newly annealed DNA molecules conjugated with biotin (40 μl) were injected into the chamber, and then were immobilized on the PEG-coated surface via biotin-neutravidin interaction by 10 min incubation, as shown in FIG. 2. Unbound DNA was washed away using T50 buffer.

### Immuno-Labeling of Immobilized DNA

After washing out the unbound DNA, the slides were incubated in blocking buffer for 30 minutes. Anti-5hmC primary antibody (cat. no. 39791, Layasan Bio.) was added to bind the 5hmC in the target DNA strand and incubated for 30 minutes. Donkey anti-Rabbit Alexa 647-labeled secondary antibody (Thermo Fisher Scientific) was added to the slides and incubated for 15 minutes.

After immuno-labelling, the subsequent single-molecule imaging was performed in imaging buffer, containing an oxygen scavenging system consisting of 0.8 mg/ml glucose oxidase, 0.625% glucose, 3 mM TROLOX® and 0.03 mg/ml catalase.

### Data Acquisition and Analysis

Single-molecule imaging was conducted by a prism-type total internal reflection fluorescence (TIRF) microscope. The excitation beam was focused into a pellin broca prism (Altos Photonics), which was placed on top of a quartz slide with a thin layer of immersion oil in between to match the index of refraction. For the TIRF microscope, a dual-laser excitation system was equipped to excite the Cy3 and Alexa 647 fluorophores. The fluorescence signals from Cy3 and Alexa 647 were detected by the electron-multiplying charge-coupled device camera (iXon 897; Andor Technology).

For 5hmC signal (Alexa 647), 20 images were stochastically recorded. Statistical analysis of spot number was performed automatically using smCamera software. Basic data analysis was carried out by the smCamera software written in C++ (Microsoft).

### Example 2. Results and Analysis

The presently disclosed method combines a selective immunofluorescent labeling strategy, single molecule fluorescent imaging technique with DNA hybridization. This technique involves denaturing dsDNA with a target DNA strand (TS) having a 5hmC modification and the annealed non-target DNA strand (NTS) are 3′ end-labeled with Cy3. A single-strand DNA probe (SP) and its complementary single-strand DNA probe (CSP) are designed and labeled with biotin and match to the TS and NTS, respectively (Table 1). During hybridization and quantification, the TS is annealed to the SP.

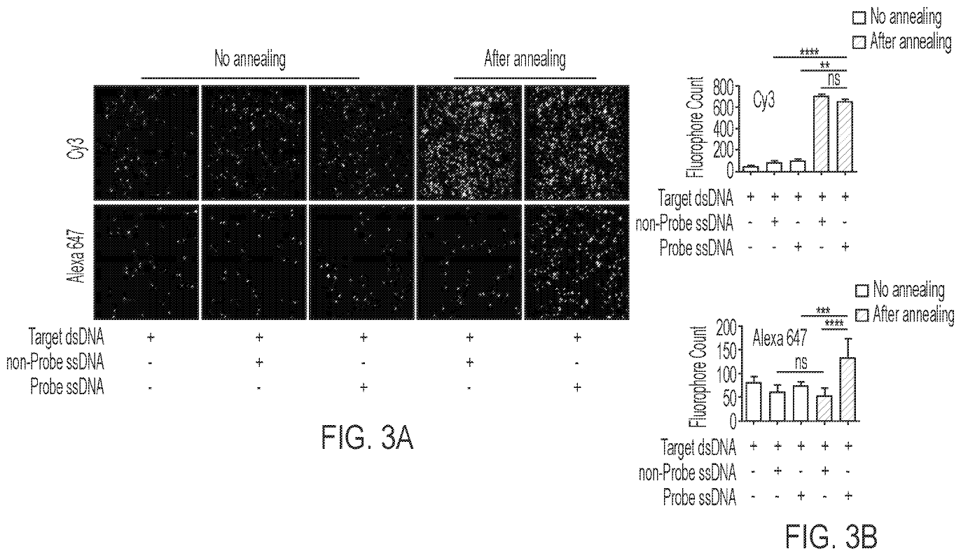

The efficiency of immobilizing freshly annealed DNA on to the PEGylated supports was evaluated by counting Cy3 fluorophores. Results are shown in FIGS. 3A and 3B. These results consist of three control groups. The three control groups are: dsDNA containing the TS and the NTS (TOnly); target dsDNA plus non-probe ssDNA without annealing (TnPOA); and target dsDNA plus probe ssDNA without annealing (TPOA). The two experimental groups were: dsDNA annealing with CSP (TnPA) and dsDNA annealing with SP (TPA).

As shown in the three control groups, few non-specific bindings of target DNA were observed. The TOnly, TnPOA, and TPOA had respective Cy3 fluorophore counts of 45.40±9.43, 83.00±16.34, and 104.0±10.92. The experimental groups had respective fluorophore counts of Cy3 of 692.90±19.92 and 654.1±17.22.

Although the fluorophore counts of Cy3 did not significantly differ between the TnPA and TPA groups, they did significantly differ between the TnPA and TnPOA groups and between the TPA and TPOA groups. As expected, both annealed TS and NTS showed similar Cy3 measurements, while the non-annealed samples did not. This indicates that the freshly annealed DNA could be specifically immobilized to the PEGylated surface.

To further test the specificity of the antibody-based assay detecting 5hmC, the fluorophore counts of the Alexa 647 signal were measured and compared to the Cy3 values, as demonstrated in FIGS. 3A and 3B.

The results included fluorophore counts of 81.15±13.51 in the Tonly group, 60.60±14.57 in the TnPOA group, 75.25±8.53 in the TPOA, 53.65±14.69 in the TnPA, and 134.7±39.46 in the TPA group. These results demonstrate that there is no significant difference between the TnPA and TnPOA groups, while in the group of TPA, the counts of the Alexa 647 label secondary antibody are significantly increased, indicating specific detection of 5hmC.

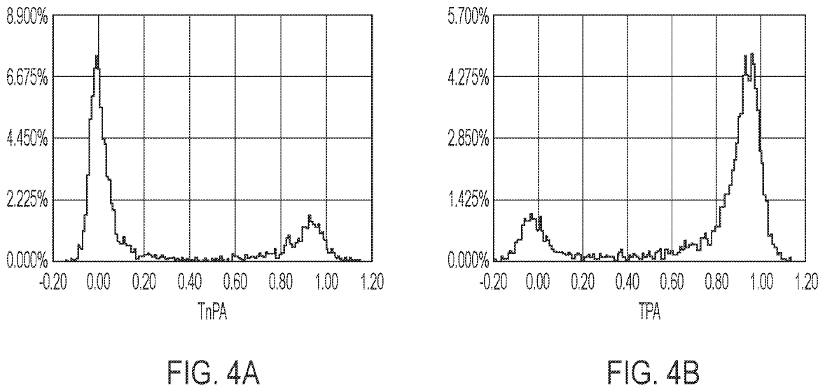

To verify the co-localizing of Cy3-labeled DNA and Alexa 647-labeled 5hmC, the FRET value in both the TnPA and TPA groups was calculated by utilizing green laser to excite Cy3 fluorophore. When the Cy3 and Alexa 647 fluorophores were close to each other, the Alexa 647 was be excited through Cy3 emission due to FRET effect. As depicted in FIGS. 4A and 4B, a high FRET value of 0.8-1.0 was only observed in the TPA group. According to those results, the method developed by combining DNA hybridization, immunofluorescence, and single-molecule imaging can specifically detect and quantify 5hmC in DNA fragments of interest.

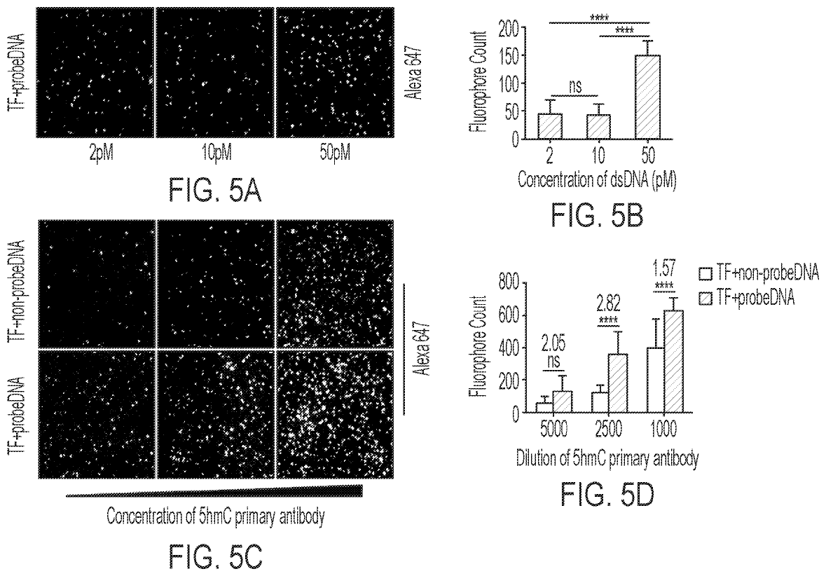

The suitable concentration of freshly annealed DNA and the primary antibody 5hmC for detection limits of this method were assessed (FIGS. 5A and 5B). Different concentrations of target dsDNA and corresponding probe ssDNA with a constant 100:1 ratio of TS to SP were annealed to obtain 2 pM, 10 pM, and 50 pM freshly annealed dsDNA.

Fluorophore counts of Alexa 647 were 46.60±24.00, 43.85±19.63, and 151.00±24.88 in the 2 pM, 10 pM, and 50 pM groups, respectively. The counts differed significantly between the 2 pM and 50 pM groups and between the 10 pM and 50 pM groups but not between the 2 pM and 10 pM groups.

The results achieved confirm that the disclosed method is highly sensitive for quantifying 5hmC, especially at the DNA concentration as low as 10 pM. The 10 pM concentration corresponds to approximately 3 pg, which is significantly lower than other 5hmC quantification methods as discussed above.

Additionally, different primary antibody concentrations were tested. As depicted in FIGS. 5C and 5D, the primary 5hmC antibody was diluted 5000, 2500, and 1000 times with a DNA concentration of 100 pM. Table 2 below depicts the relative fluorophore counts.

TABLE 2
Fluorophore counts with varying concentrations
of primary antibody.

| | | Alexa 647 | Alexa 647 | |
|---|---|---|---|---|
| | | Fluorophore | Fluorophore | Ratio of |
| | | Signal TPA | Signal TnPA | Alexa |
| | | (Presence of | (Presence of | fluorophore |
| Anti-5 hmC dilution | | 5 hmC) | 5 hmC) | signaling |
| 5000 | (.2 ng/uL) | 134.70 ± 90.73 | 65.75 ± 33.63 | 2.05 |
| 2500 | (.4 ng/uL) | 363.70 ± 135.24 | 128.75 ± 40.27 | 2.82 |
| 1000 | (1 ng/uL) | 631.65 ± 77.74 | 402.45 ± 175.78 | 1.57 |

As can be noted, fluorophore counts of Alexa 647 increased as the concentration of the primary antibody increased in both the TPA and TnPA groups, however the background also increased. This lead to a decreased ratio of TPA to TnPA, indicating that when the DNA concentration is 100 pM, a primary antibody concentration dilution of 2500 times is optimal.

### Example 3. Diagnosis of Cancer by SMII Analysis

First, single-stranded DNA probes complementary to known genomic regions containing epigenetic modifications associated with a type of cancer are designed. A sample containing cfDNA or genomic DNA is obtained from a patient suspected of having the type of cancer. The cfDNA is labeled and imaged according to the disclosed SMII methods to localize and quantify the DNA epigenetic modifications in the patient's DNA and generate an epigenetic profile. The patient's epigenetic profile is compared to a reference epigenetic profile for the type of cancer assessed. When the patient's epigenetic profile and the reference epigenetic profile are substantially concordant, the patient is diagnosed with cancer. The method may further be used to assess progress and stage of cancer, using external and internal controls.

In summary, the methods disclosed herein relate to a sensitive, low-input assay of quantifying epigenetic modifications in DNA fragments at the single-molecule level. Combining DNA hybridization, immunofluorescent imaging, and TIRF microscopy, the methods disclosed here in can reveal which gene the modifications are located in and further provide information for quantifying the epigenetic modifications. In general, speed, maneuverability, and sensitivity are the three key factors assessed to evaluate the clinical applicability of a laboratory technology. Along those lines, the methods allows quantifying epigenetic modification in DNA oligonucleotides in the clinic in less than 6 hours.

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A method for quantifying epigenetic modifications in DNA, the method comprising:
(a) providing a target DNA strand comprising at least one epigenetic modification;
(b) annealing a single-stranded DNA probe to the target DNA strand, wherein the probe is conjugated to a biotin moiety;
(c) immobilizing the annealed DNA on a support;
(d) contacting the immobilized DNA with a primary antibody that binds to the at least one epigenetic modification;
(e) contacting the immobilized DNA with a secondary antibody, wherein the second antibody is labeled with a fluorophore and wherein the secondary antibody binds to the primary antibody; and
(f) detecting the fluorophore using prism-based single molecule total internal reflection fluorescence (TIRF) microscopy.

2. The method according to claim 1, wherein the target DNA strand is annealed to a non-target DNA strand.

3. The method according to claim 2, further comprising denaturing the target DNA strand and the non-target DNA strand.

4. The method according to claim 1, wherein the at least one epigenetic modification is selected from the group consisting of 5-hydroxymethylcytosine (5hmC), 5-methylcytosine (5mC), 5-formylcytosine (5fC), and 5-carboxylcytosine (5caC).

5. The method according to claim 1, wherein the epigenetic modification is associated with cancer.

6. The method according to claim 1, wherein the support comprises a surface-tethered moiety selected from the group consisting of avidin, streptavidin, and neutravidin.

7. The method according to claim 1, wherein the annealed DNA is immobilized via avidin-biotin pairing.

8. The method according to claim 1, wherein the support comprises a polymer-coated quartz surface.

9. The method according to claim 1, wherein the DNA is selected from the group consisting of genomic DNA and cell-free DNA (cfDNA).

10. The method according to claim 1, further comprising quantifying a number of epigenetic modifications of the target DNA strand.

11. The method according to claim 1, wherein the fluorophore is selected from the group consisting of Cy3, Cy5, Quasar 570, Quasar 670, Alexa Fluor 555, Alexa Fluor 647, BODIPY V-1002, BODIPY V-1005, POPO-3, TOTO-3, PO-PRO-3, and TO-PRO-3.

12. A method of diagnosing cancer in a subject suspected of having cancer, the method comprising:
(a) providing a biological sample from the subject, the sample comprising a target DNA strand comprising a least one epigenetic modification, wherein the target DNA strand is annealed to a non-target DNA strand;
(b) annealing a single-stranded DNA probe to the target DNA strand, wherein the probe is conjugated to a biotin moiety;
(c) immobilizing the annealed DNA on a support;
(d) contacting the immobilized DNA with a primary antibody that binds to the at least one epigenetic modification;
(e) contacting the immobilized DNA with a secondary antibody, wherein the second antibody is labeled with a fluorophore and wherein the secondary antibody binds to the primary antibody;
(f) detecting the fluorophore using prism-based single molecule total internal reflection fluorescence (TIRF) microscopy based on the fluorophore detection;
(g) quantifying a number of epigenetic modifications in the target DNA strand;
(f) comparing the number of epigenetic modifications to a reference epigenetic profile for cancer; and
(h) diagnosing the subject as having cancer when the quantifying of step (g) correlates with the reference epigenetic profile for cancer.

13. The method according to claim 12, wherein the fluorophore is selected from the group consisting of Cy3, Cy5, Quasar 570, Quasar 670, Alexa Fluor 555, Alexa Fluor 647, BODIPY V-1002, BODIPY V-1005, POPO-3, TOTO-3, PO-PRO-3, and TO-PRO-3.

14. The method according to claim 12, wherein the epigenetic modification is selected from the group consisting of 5-hydroxymethylcytosine (5hmC), 5-methylcytosine (5mC), 5-formylcytosine (5fC), and 5-carboxylcytosine (5caC).

15. The method according to claim 12, wherein the support comprises a surface-tethered moiety selected from the group consisting of avidin, streptavidin, and neutravidin.

16. The method according to claim 12, wherein the target DNA strand is immobilized via avidin-biotin pairing.

17. The method according to claim 12, wherein the support comprises a polymer-coated quartz surface.

18. The method according to claim 12, wherein the target DNA strand is selected from the group consisting of genomic DNA and cell-free DNA (cfDNA).

19. The method according to claim 12, wherein the biological sample is selected from the group consisting of blood, serum, plasma, urine, tissue, and cultured cells.

20. The method according to claim 12, further comprising treating the diagnosed subject with a therapeutic agent specific for the cancer.
